(19) United States
(12) Patent Application Publication
(10) Pub. No.: US 2021/0088542 A1
(43) Pub. Date: Mar. 25, 2021

(54) EQUIPMENT FOR THE TRACEABILITY OF BIOLOGICAL SAMPLES

(71) Applicant: DREAMPATH DIAGNOSTICS, Strasbourg (FR)

(72) Inventors: Pablo JORDAN NAVAS, Madrid (ES); Valerie WILHELM, Eschau (FR)

(21) Appl. No.: 17/021,680

(22) Filed: Sep. 15, 2020

(30) Foreign Application Priority Data
Sep. 19, 2019 (FR) 19 10339

(51) Int. Cl. G01N 35/00; B01L 3/00; G16B 40/00; G16B 50/00; G16H 10/60

(52) U.S. Cl. CPC G01N 35/00871; B01L 3/545; G01N 35/00029; G01N 35/00732; G16B 40/00; G16B 50/00; G01N 2035/0091; G01N 2035/00138; G01N 2035/00752; G01N 2035/00831; G01N 2035/00841; G01N 2035/00881; G16H 10/60

(57) ABSTRACT

The equipment for the traceability of biological samples placed in cassettes provided with encoded identification data and intended to be stored within baskets, the general shape of which may vary from one basket to another includes at least one container to accommodate at least one basket, at least one apparatus for reading encoded data affixed on cassettes arranged in at least one basket placed in the container, a device for computerized processing of the data read by the apparatus for reading, and a device for identifying the shape of the basket placed in the container.

### CROSS-REFERENCE TO RELATED APPLICATIONS

See Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

### 1. Field of the Invention

The present invention relates to an equipment item for the traceability of biological samples placed in inclusion cassettes.

### 2. Description of Related Art Including Information Disclosed Under 37 CFR 1.97 and 37 CFR 1.98.

In the context of the medical care of a patient or of research work in the human, animal or plant fields, tissue or cell samples are commonly taken for histological and/or molecular analysis, which may or may not be intended to prepare a diagnosis.

In order to allow perfect viewing under the microscope of the cell or tissue structures being analyzed, such samples are subject to prior preparation including different steps.

In short, during the latter steps, the fresh specimens, initially kept in specimen containers filled with formalin, are placed in open inclusion cassettes, to undergo a dehydration phase, then a step for inclusion in paraffin resulting in blocks of biological samples. Fine and regular cuts, taken from the latter, are next placed on microscope slides, undergo various treatments intended to dye the structural elements to be observed, and are lastly covered with a resin, then a strip allowing them to be read under the microscope and providing them with protection.

All of these steps to prepare the biological specimens for analysis assume many manipulations, during which measures to ensure traceability must be taken.

Solutions have already been proposed to that end, in particular to ensure automated traceability of biological specimens stored in blocks of paraffin or on microscope slides.

However, it has been observed that to date, no streamlined solution is currently available to effectively and reliably manage the biological specimens placed in inclusion cassettes for the dehydration thereof.

Indeed, the existing equipment has been developed for very specific applications, and depending on the case is appropriate for monitoring paraffin blocks only or for monitoring microscope slides only.

In short, no apparatus dedicated to monitoring inclusion cassettes has been proposed at this time, such that the operations necessary for the traceability of biological specimens stored in such containers must, at this stage, still be done manually.

Conventionally, each biological sample is assigned a unique reference number, supplied by the computer system of the establishment having collected it. This number connects it to the patient from which it originates and will follow it all the way to its destruction, whether it is stored in a specimen container, an inclusion cassette, a paraffin block, or on a microscope slide, in the form of encoded data of the barcode, matrix code, RFID chip type, affixed on the latter during its preparation.

Currently, when freshly collected samples are handled by the appropriate analysis departments, they are recorded in a database, from a manual reading of the encoded data affixed on the formalin jars containing them. More specifically, at the end of this step, the database contains information for each sample such as the identity of the patient, the nature of the sample and the number of cassettes to be prepared therefrom. The samples are next removed from their container one after the other, subjected to a first visual examination, then placed individually in open cassettes bearing their identification data and allowing them to be exposed to various treatments intended to replace the water contained in their cells with a resin. The steps of this process are commonly simultaneously applied to a plurality of biological samples using dedicated apparatuses, called “tissue processor”. To that end, the cassettes in which they are contained are grouped together in baskets provided with these same apparatuses, facilitating their handling. For traceability purposes, when the baskets are filled with cassettes, it is common practice to verify manually, using the database initially filled in, whether the number of cassettes present in each basket for the “tissue processor” indeed corresponds to the number of cassettes meant to have been prepared. If there is a difference, an error search can thus be done, in order to find any cassette that may have been forgotten on a lab table with its content, or to ensure that all of the samples received by the laboratory have been placed in the cassettes and therefore processed. Because the storage time of the specimens in the formalin is very short, such an inspection is indeed essential to avoid any untimely loss of material.

Clearly, such a manual approach is particularly tedious and time-consuming, inasmuch as it assumes several manipulations of each biological sample to read and record its identification data during the successive steps mentioned above, and to compare the information relative to these same steps appearing in the database.

Additionally, due to the aging of the population and the increased incidence rate of certain diseases, in particular cancer, the number of specimens arriving daily in histopathology laboratories is continuing to increase. Such a situation inevitably leads to an increased workload for laboratory personnel, who must deplore the obsolescence of the manual traceability methods currently available to them to perform part of their tasks.

### BRIEF SUMMARY OF THE INVENTION

As a result, the present invention aims to propose a solution making it possible to improve the productivity of all of the personnel who may participate during the preparation, analysis and storage phases of tissue or cell samples owing to an automation of the traceability of biological specimens during the entire preparation and analysis process that they undergo, including during the dehydration step, in order to reduce the workload of the operators, avoid any risk of error and loss of material, and guarantee the establishment of reliable diagnostics attributed to the correct patient.

Furthermore, the invention also aims to propose an equipment item compatible with the use of potentially toxic and/or corrosive products, and having a structure making it possible to limit the exposure to these same products by the operators called upon to handle them and to protect its component elements.

To that end, the present invention relates to an equipment item for the traceability of biological samples placed in inclusion cassettes provided with identification information in the form of encoded data and intended to be stored next to one another in housings arranged within baskets, the general shape of which may vary from one basket to another, said equipment item including:
at least one container designed to be able to accommodate at least one basket,
at least one apparatus for reading encoded data affixed on cassettes arranged in at least one basket placed in the container, said apparatus being delimited by a bottom, a peripheral wall and an upper wall and being provided with at least one opening for access to at least one inner cavity configured to accommodate the container and equipped with means for reading encoded data,
means for computerized processing of the data read by the reading means, and
means for identifying the shape of the basket(s) placed in the container.

According to one feature of the equipment item according to the invention, the container is subdivided into several compartments having different shapes, each configured to receive a basket of a given shape, while the reading apparatus is equipped with detection means able to detect the presence, or if applicable, the absence of a basket in at least one of said compartments of said container and to send the corresponding information to means for controlling the means for reading encoded data.

Furthermore, it is also provided that the container preferably includes an open-worked bottom.

According to another additional feature, the cavity of the apparatus can include support means for a container, molded to allow an incline of the bottom of the container relative to the bottom of the apparatus. The incline has the advantage of favoring the flow of the formalin impregnating the specimen contained in the cassettes, and therefore facilitating the reading of the data affixed on the latter.

To that end, a variant embodiment has been conceived in which the peripheral wall of the apparatus includes two opposite walls provided with rails each forming an angle α, preferably inclusively between 10° and 80°, with the bottom of the apparatus, said rails being designed to be able to cooperate with slides included by said container.

Additionally, the equipment item according to the invention is further characterized in that it may include a tray configured to rest on its bottom.

Moreover, one additional feature of the equipment item according to the invention is defined by the fact that the means for reading encoded data included by said reading apparatus are designed to be able to withstand a moist atmosphere.

At least one fan installed in the cavity of said apparatus can further be provided, in order to actively discharge the formalin vapors risking preventing the reading of the encoded data.

According to the invention, the means for reading the encoded data can include a light scanner and/or a radio scanner of the RFID type.

### BRIEF DESCRIPTION OF THE SEVERAL VIEWS OF THE DRAWINGS

The appended drawings illustrate the invention.

FIGS. 1A. 1B, and 1C show schematic views of baskets for storing inclusion cassettes during the step for dehydration of the biological samples, showing different structures.

FIG. 2 shows a top plan view of a variant embodiment of a container according to the invention.

FIG. 3 illustrates a sectional view of the equipment item according to the invention.

### DETAILED DESCRIPTION OF THE INVENTION

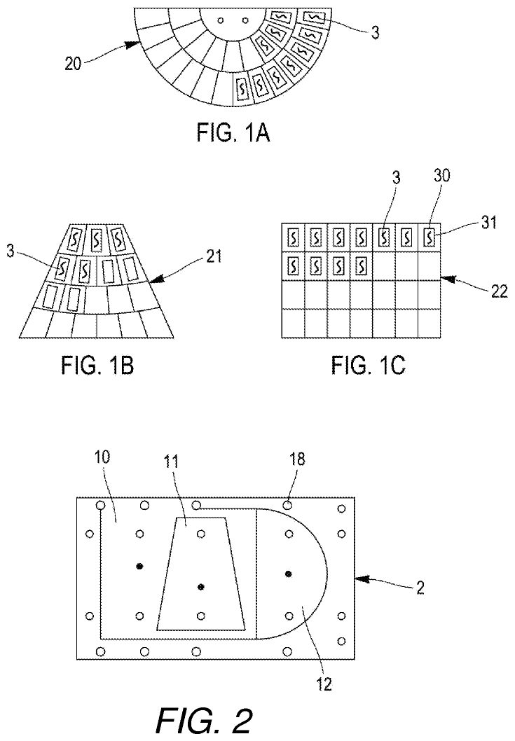

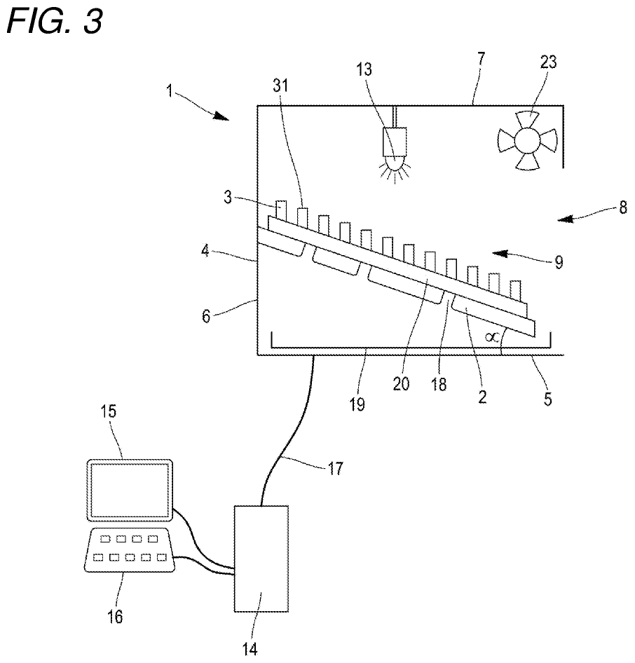

In reference to these drawings, in the illustrated variant embodiment, the equipment item 1 according to the invention includes a universal container 2 designed to be able to accommodate different types of baskets 20, 21, 22 for inclusion cassettes 3. Conventionally, such baskets 20, 21, 22 are provided with the “tissue processors” used to dehydrate the specimens, and have a structure whose shape may vary, from one basket model to another. In short, the shape of the baskets is defined by their manufacturer, and may for example correspond to a half-moon shape (cf. FIG. 1A), a trapezoidal shape (cf. FIG. 1B), a parallelepipedal shape (cf. FIG. 1C), or any other shape making it possible to store a plurality of inclusion cassettes 3 in a sequenced manner. As shown in FIGS. 1A to 1C, the latter are generally positioned in individual housings included by the baskets 20, 21, 22, such that their face 30 provided with encoded identification data 31 is visible and preferably oriented upward.

In reference to FIG. 2, it has been imagined in the context of the invention to design the container 2 such that it includes different compartments with different shapes, each dedicated to receiving a basket 20, 21, 22 with a given structure or shape. Thus, in the example illustrated in FIG. 2, the container 2 assumes the form of a plate, the bottom of which preferably has a plurality of apertures 18, including three compartments 10, 11, 12 respectively dedicated to receiving a basket 22 of parallelepipedal shape, a trapezoidal basket 21 and a half-moon shaped basket 20. The receiving zones 10 and 11 overlap; of course, such a container is not able to simultaneously accommodate a parallelepipedal basket 22 and a trapezoidal basket 21. Conversely, it is perfectly suited to simultaneously receiving a parallelepipedal basket 22 or a trapezoidal basket 21 and a half-moon shaped basket 20. In other words, such a container 2 may, depending on the case, accommodate a single basket 10, or 11, or 12, or two baskets 10 and 12 or 11 and 12. Of course, an arrangement of the compartments other than that included by the container 2 of the illustrated example may also be provided.

Furthermore, the equipment item 1 also includes an apparatus 4 for reading encoded data affixed on the cassettes 3 placed in at least one of the baskets 20, 21, 22 placed in a container 2. More specifically, the apparatus 4 is delimited by a bottom 5, a peripheral wall 6, and an upper wall 7. It is provided with at least one opening 8 for access to at least one inner cavity 9 configured to accommodate a container 2 and equipped with means 13 for reading encoded data, for example a light scanner or a radio scanner of the “RFID” type. Means for computerized processing of the data read by the reading means 13, such as a computer 14 comprising a screen 15 and a keyboard 16, are also connected to the apparatus 4 by a wired or wireless connection 17.

In the illustrated variant embodiment, the reading apparatus 4 is further equipped with means (not illustrated) for detecting the presence of at least one basket 20, 21, 22 in the container 2, and designed to be able to send the corresponding information to means for controlling the means for reading encoded data 31. In short, such a feature makes it possible to program automatically, following a simple detection of the presence of a basket in a given compartment, the reading means such that they are able to suitably read the identification data 31 affixed on inclusion cassettes 3 placed depending on the case in a half-moon shaped basket 20, or in a trapezoidal basket 21, or in a parallelepipedal basket 22. Owing to such a structure, the equipment item 1 makes it possible to automate the reading of the encoded data 31 included by the cassettes 3 and to verify all of the content of one or two baskets 20, 21 or 22 simultaneously in a single step, irrespective of the general shape of such baskets.

Furthermore, in the illustrated variant embodiment, the peripheral wall 6 of the apparatus 4 includes two opposite walls provided with rails each forming an angle α, preferably inclusively between 10° and 80°, with the bottom 5 of the apparatus 4 and designed to be able to cooperate with slides included by the container 2. Such a structure favors any flow of formalin, with which the biological samples contained in the inclusion cassettes 3 may still be impregnated, through the apertures 18 of the container 2. A removable tray 19, molded to rest on the bottom 5 of the apparatus 4, further makes it possible to recover and discharge any liquid flowing from the container 4, so as to avoid any stagnation of toxic product in the apparatus 4 and to favor a suitable reading of the encoded data 31.

In this respect, it should be noted that in order to improve the longevity of the apparatus 4 and to favor suitable reading of the encoded data 31, at least one fan 23 is installed in the cavity 9, while the means 13 for reading encoded data are preferably configured to withstand a humid and potentially corrosive atmosphere.

1. An equipment item for traceability of biological samples placed in inclusion cassettes provided with identification information comprised of encoded data and stored next to one another in housings arranged within baskets, the general shape of which may vary from one basket to another, said equipment item comprising:
at least one container to accommodate at least one basket,
at least one apparatus for reading encoded data affixed on cassettes arranged in at least one basket placed in the container, said apparatus being comprised of a bottom, a peripheral wall, an upper wall and with at least one opening for access to at least one inner cavity to accommodate the container and equipped with means for reading encoded data,
means for computerized processing of the data read by the reading means, and
means for identifying the shape of the basket placed in the container.

2. The equipment item according to claim 1, wherein the container is subdivided into several compartments having different shapes, each configured to receive a basket of a given shape, while the reading apparatus is comprised of a detection means for the presence, or if applicable, the absence of a basket in at least one of said compartments of said container and to send the corresponding information to means for controlling the means for reading encoded data.

3. The equipment item according to claim 2, wherein the bottom of the container is open-worked.

4. The equipment item according to claim 1, wherein the cavity of the apparatus comprises support means for a container, molded to allow an incline of the bottom of the container relative to the bottom of the apparatus.

5. The equipment item according to claim 4, wherein the peripheral wall of the apparatus comprises two opposite walls provided with rails each forming an angle α with the bottom of the apparatus and being cooperative with slides included by said container.

6. The equipment item according to claim 5, wherein the angle α is inclusively between 10° and 80°.

7. The equipment item according to claim 1, wherein the apparatus comprises a removable tray molded to rest on its bottom.

8. The equipment item according to claim 1, wherein the means for reading encoded data of said reading apparatus withstand a moist atmosphere.

9. The equipment item according to claim 1, wherein said apparatus is comprised of at least one fan installed in said cavity.

10. The equipment item according to claim 1, wherein the means for reading the encoded data are defined by at least one of a group consisting of: a light scanner and a radio scanner of the RFID type.
